(19) United States
(12) Patent Application Publication
(10) Pub. No.: US 2010/0174197 A1
(43) Pub. Date: Jul. 8, 2010

(54) BIOLOGICAL INFORMATION IMAGING APPARATUS

(75) Inventors: Takao Nakajima, Kawasaki-shi (JP); Kazuhiko Fukutani, Yokohama-shi (JP); Yasufumi Asao, Atsugi-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/602,715

(22) PCT Filed: Jun. 20, 2008
(86) PCT No.: PCT/JP2008/061684

§ 371 Date: Dec. 2, 2009

(30) Foreign Application Priority Data
Jun. 22, 2007 (JP) 2007-164654

(51) Int. Cl. A61B 6/00

(52) U.S. Cl. 600/478

(57) ABSTRACT

A living body information imaging apparatus includes a member that is adapted to be introduced into an interior of a living body and has a light irradiation portion for irradiating light with an angle of irradiation equal to or more than 2π steradian to the interior of the living body, and a signal detector that is arranged outside the living body and detects a signal output based on light irradiation in the interior of the living body by the member having the light irradiation portion.

### TECHNICAL FIELD

The present invention relates to a biological information imaging apparatus, and more particularly to a biological information imaging apparatus using an optoacoustic (or photoacoustic) effect.

### BACKGROUND ART

In recent years, there has been proposed photoacoustic imaging that serves to obtain an optical property distribution in the interior of a living body with a high resolution by using a property of an ultrasonic wave which is less scattered in the interior of the living body as compared with light (a first, a second and a third patent document to be described later).

In this method, pulsed light generated from a light source is irradiated to the living body, and an acoustic wave generated from a tissue of the living body that has absorbed the energy of the pulsed light being propagated and diffused therein is detected, whereby it is possible to obtain an optical property distribution in the living body by performing analytic processing on the detection signal of the acoustic wave.

In addition, in the third patent document, it is stated that the optical property distribution in the interior of a living body can be obtained by detecting an ultrasonic wave, which is generated in the interior of the living body by the irradiation of pulsed light, by means of a plurality of transducers.

[First Patent Document] U.S. Pat. No. 4,385,634

[Second Patent Document] U.S. Pat. No. 5,840,023

[Third Patent Document] U.S. Pat. No. 5,713,356

### DISCLOSURE OF INVENTION

However, in the photoacoustic imaging, the sound pressure of an acoustic wave obtained from an absorber in the living body due to optical absorption is proportional to the local amount of light that has arrived at the absorber.

The light irradiated to the living body is rapidly attenuated due to scattering and absorption thereof in the interior of the living body, so the sound pressure of the acoustic wave generated in a deep tissue of the living body attenuates greatly in accordance with the distance thereof from the place of light irradiation.

From these, it becomes difficult to obtain information on a deep interior of a living body with an apparatus that irradiates light to the living body from the outside thereof and detects an acoustic wave from the living body at a location outside thereof, as described in the first patent document.

Accordingly, in the second patent document, there is presented a photoacoustic imaging apparatus that has a light irradiation point and a transducer provided in an endoscope in the photoacoustic imaging of an internal organ such as an artery. Thus, by performing light irradiation and acoustic wave detection in the vicinity of the internal organ in the form of a specimen or an object to be tested in a deep portion of a living body, it is made possible to effect the optical property distribution imaging of the internal organ in the deep portion of the living body.

However, in such a method disclosed in the second patent document, there arises a problem that the propagation area of light is limited, so light can be irradiated only to an absorber that exists in a specific region.

In addition, with the transducer arranged in the endoscope, there also arises another problem that due to constraints of the apparatus, it is difficult to detect all the acoustic wave which is propagated from various directions, and hence it is also difficult to effect the imaging of an optical property distribution over a wide range.

In view of the problems as referred to above, the present invention is intended to provide a biological information imaging apparatus and a biological information imaging method which are capable of obtaining an optical property distribution in a deep portion of a living body over a wide range.

A living body information imaging apparatus of the present invention comprises a member that is adapted to be introduced into an interior of a living body and has a light irradiation portion for irradiating light with an angle of irradiation equal to or more than 2π steradian to the interior of the living body, and a signal detector that is arranged outside the living body and detects a signal output based on light irradiation in the interior of the living body by the member having the light irradiation portion.

According to the present invention, it is possible to obtain an optical property distribution in a deep portion in the interior of a living body.

Further features of the present invention will become apparent from the following description of exemplary embodiments with reference to the attached drawings.

### BRIEF DESCRIPTION OF DRAWINGS

FIG. 1 is a view for explaining a construction example of a biological information imaging apparatus in a first embodiment of the present invention;

FIG. 2 is a view for explaining a construction example of a biological information imaging apparatus in a second embodiment of the present invention;

FIG. 3 is a view for explaining an insertion type light source that is used with a biological information imaging apparatus in a first example of the present invention; and

FIG. 4 is a view for explaining an insertion type light source having a puncturing function that is used with a biological information imaging apparatus in a second example of the present invention.

### DESCRIPTION OF EMBODIMENTS

By using the construction of the present invention, it becomes possible to obtain an optical property distribution in a deep portion of the interior of a living body over a wide range in the following manner.

First of all, in a step of irradiating light in the interior of the living body, a member with a light irradiation portion having a light irradiation range equal to or more than 2π steradian (symbol: sr) is introduced into the interior of the living body, and light is irradiated in the interior of the living body by means of the light irradiation portion.

Here, the “steradian (symbol: sr)” is the unit of solid angle in the International System of Units (SI), and corresponds to the radian of plane angle.

Then, in a step of detecting a signal by means of a signal detector (acoustic wave detector), the signal (elastic wave), which is output based on the light irradiation of the member having the light irradiation portion, is detected at multiple points in the following manner.

That is, the signal (elastic wave) is detected at the multiple points by means of a plurality of transducers that are arranged outside the living body or by scanning the transducers.

Thus, it becomes possible to effect imaging of an optical property value distribution in the deep portion of the interior of the living body over a wide range thereof, and it also becomes possible to obtain a large acoustic wave signal by performing appropriate acoustic impedance matching between the living body and the transducers.

Further, since light can be irradiated in the vicinity of a test object or specimen lying deep in the living body, it also becomes possible to effect imaging of the optical property of a tissue of the living body by using wavelength regions other than a near-infrared region where absorption in the living body is strong.

Now, embodiments of the present invention will be further described while referring to the accompanying drawings.

### FIRST EMBODIMENT

First, reference will be made to a biological information imaging apparatus in a first embodiment of the present invention.

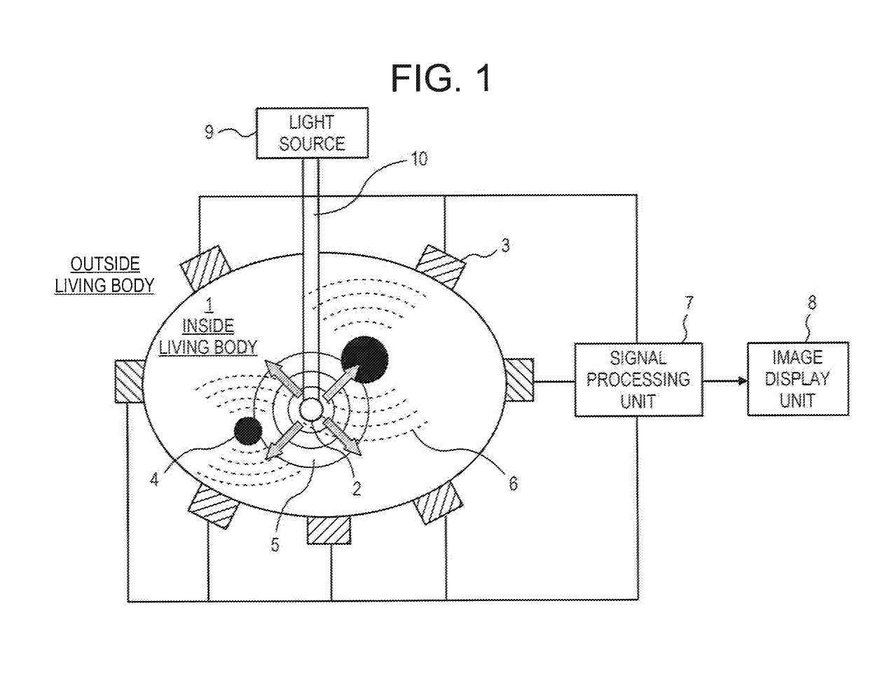

FIG. 1 shows a view for explaining a construction example of the biological information imaging apparatus of the first embodiment.

In FIG. 1, reference numeral 1 denotes a living body, 2 a light irradiation point, 3 acoustic wave detectors, 4 a light absorber, 5 light propagating in the interior of the living body, 6 an acoustic wave, 7 a signal processing unit (information processing unit), 8 an image display unit, 9 a light source, 10 an optical wave guide (optical fiber) that is connected to the light source 9.

The biological information imaging apparatus of this first embodiment serves to make it possible to provide the imaging of an optical property distribution in a living body as well as a concentration distribution of materials that constitute a tissue of the living body obtained from the distribution information in order to perform diagnoses on tumors, blood vessel diseases, etc., as well as observation of the follow-up of a chemical treatment or the like.

The biological information imaging apparatus of this embodiment is provided with the light irradiation point 2 which is arranged in the interior of the living body 1 and at which light is irradiated to the living body 1.

In addition, the biological information imaging apparatus also has the plurality of acoustic wave detectors 3 arranged outside the living body 1, each of the detectors 3 serving to detect an acoustic wave 6, which the light absorber 4 in the living body such as a tumor, a blood vessel or the like in the living body generates by absorbing a part of the optical energy of the light, and to convert it into a corresponding electric signal.

Further, the biological information imaging apparatus is provided with the signal processing unit 7 that obtains optical property distribution information by analyzing the electric signals from the acoustic wave detectors 3.

The light irradiation point 2 is used as a means for irradiating light of a specific wavelength that is to be absorbed by a specific one of components that constitute the living body 1.

The light emitted from the light source 9 outside the living body 1 propagates through the optical wave guide 10 such as an optical fiber or the like, which is coupled to a catheter, so that it is irradiated from the light irradiation point 2 in the living body 1. The optical wave guide 10 is characterized in that it is provided at its distal end with a medium that serves to expand the irradiation or emission angle of the light to an angle equal to or more than 2π steradian, and this medium becomes the light irradiation point 2.

As such a medium, there can be used a light scattering medium, a movable type mirror or the like.

In addition, as the light scattering medium, there can be used, for example, spherical frosted or ground glass or the like.

A light source that generates pulsed light can be used as the light source 9.

The pulsed light has a pulse interval on the order of few ns to few hundred ns, and it is preferable that the wavelength of the pulsed light be not less than 400 nm and not more than 1,600 nm.

Laser is preferable as the light source 9, but it is also possible to use a light emitting diode or the like, instead of laser.

A variety of kinds of lasers such as a solid state laser, a gas laser, a dye laser, a semiconductor laser and the like can be used as the laser light source.

Here, note that if coloring matter (or dyestuff) or OPO (Optical Parametric Oscillators) with a convertible or variable oscillating wavelength can be used, it will be possible to measure the variation or difference in the optical property distribution according to the wavelength.

It is preferred that the wavelength of the light source 9 being used be in a region from 700 nm to 1,100 nm in which the absorption of light in the interior of the living body 1 is limited.

Since the light irradiation point is arranged in the interior of the living body 1, it is possible to use a wavelength range, such as for example 400 nm to 1,600 nm, wider than the above-mentioned wavelength range, and in addition, to use additional wavelength ranges including terahertz waves, micro waves, and radio waves.

If the light irradiated from the light irradiation point 2 to the living body 1 can act on the tissue of the living body 1 to generate a modulated signal, which can be acquired by the signal detectors 3 of this first embodiment, it is possible to obtain an optical property distribution in the interior of the living body 1 from the signal, so any kind or type of signal detectors can be used for such a purpose. As such signal detectors, there can be used photo detectors such as photo multipliers, photo diodes or the like, acoustic wave detectors that are capable of detecting acoustic signals, other various kinds of detectors that are capable of detecting electromagnetic waves, heat, etc. In particular, the acoustic wave detectors are preferably used from the viewpoint that they are the same as acoustic wave detectors of ultrasonic diagnostic apparatuses widely used in the past, and that it is easy to convert signals obtained by them into image information. Each of the acoustic wave detectors serves to detect the acoustic wave (elastic wave) generated from the light absorber in the living body having absorbed a part of the energy of the light irradiated from the light irradiation point source to the living body, and to convert it into a corresponding electric signal.

There can be used any acoustic wave detectors such as transducers using a piezo-electric phenomenon, transducers using the resonance of light, transducers using a change in capacity, as long as they are capable of detecting an acoustic wave signal.

Moreover, although in this embodiment, there has been shown the case where the plurality of acoustic wave detectors are arranged on a surface of the living body, the present invention is not limited to such an arrangement, but may only include a construction that the acoustic wave can be detected in a plurality of locations. Specifically, as long as the acoustic wave can be detected in a plurality of locations, the same effects can be obtained, so a single acoustic wave detector may be scanned on the living body surface.

Here, note that when an electric signal obtained from an acoustic wave detector is small, it is preferable that the strength of the signal be amplified by the use of an amplifier.

Moreover, it is also desirable that an acoustic impedance matching agent for suppressing the reflection of acoustic or sound waves be used between the acoustic wave detectors and a material of the living body to be measured.

The signal processing unit 7 of this embodiment serves to analyze the electric signals from the acoustic wave detectors 3, so that optical property distribution information on the living body can be thereby obtained.

For example, as shown in FIG. 1, the signal processing unit 7 calculates, based on the electric signals obtained from the acoustic wave detectors 3, the position and size of the light absorber in the interior of the living body, and calculates an optical property distribution such as a light absorption coefficient distribution or a light or optical energy accumulation amount distribution, or the like.

Here, note that as the signal processing unit 7, there can be used anything that is able to store the strength and its change over time of the acoustic wave, and to convert them into data for an optical property distribution by a calculation means.

As such, there can be used, for example, an oscilloscope and a computer that can analyze data stored in the oscilloscope.

In case where light having a plurality of wavelengths is used, optical coefficients in the interior of the living body are calculated for each wavelength, and the values of the optical coefficients thus calculated are compared with the intrinsic wavelength dependences of materials (glucose, collagen, oxidized and reduced hemoglobin, etc.) that constitute the tissue of the living body.

As a result, it is also possible to image the concentration distribution of the materials that constitute the living body. In addition, in this embodiment of the present invention, it is desirable that an image display unit 8 be provided for displaying the image information obtained by the signal processing.

The photoacoustic imaging of the deep portion in the interior of the living body is made possible by the use of the biological information imaging apparatus shown in this embodiment.

On the other hand, in the above-mentioned second patent document, there is presented a photoacoustic imaging apparatus that has a light irradiation point and a transducer provided in an endoscope in the photoacoustic imaging of an internal organ such as an artery. In this case, there is the following problem. That is, since the transducer is arranged in the interior of the living body, it becomes difficult to take matching of acoustic impedance, thus making it difficult to obtain a large signal.

On the other hand, with the use of the member, which is adapted to be introduced into the interior of the living body and has the light irradiation portion for irradiating light to the interior of the living body, and the signal detectors arranged outside the living body, it is possible to use an acoustic impedance adjustment layer between the signal detectors and the living body. As a result, a large signal can be obtained in comparison with the case in which the signal detectors are arranged in the living body.

### SECOND EMBODIMENT

Reference will be made to a biological information imaging apparatus according to a second embodiment of the present invention.

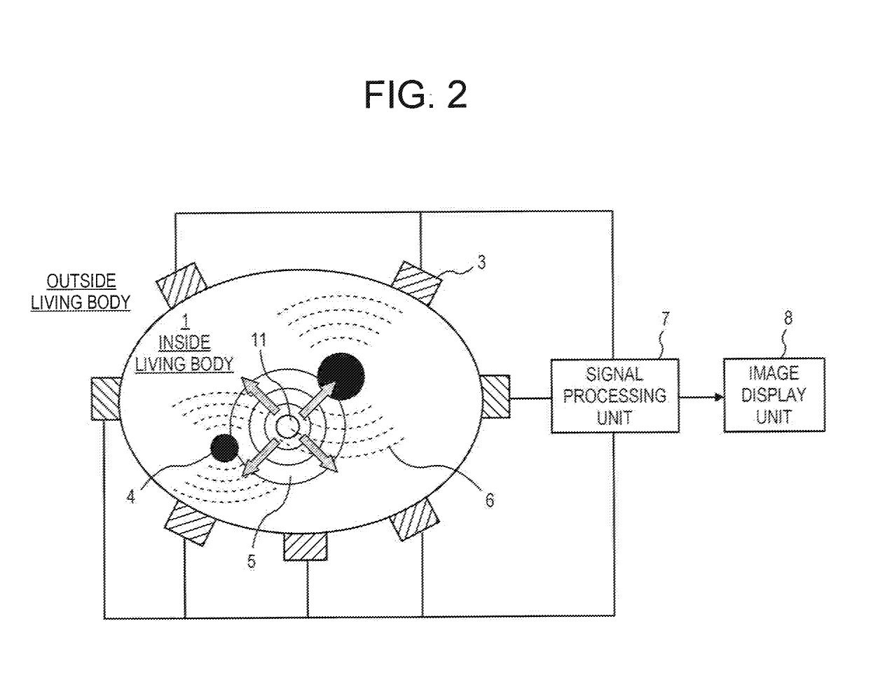

FIG. 2 shows a view for explaining a construction example of the biological information imaging apparatus of this second embodiment.

In FIG. 2, those portions of this second embodiment which are identical in construction to the first embodiment shown in FIG. 1 are denoted by the same symbols as in the first embodiment, while omitting the explanation of the identical or common parts.

In FIG. 2, reference numeral 11 denotes a light irradiation point that is arranged in the interior of a living body 1 for irradiating light to the living body 1.

The light irradiation point 11 is used as a means for irradiating light of a specific wavelength that is to be absorbed by a specific one of components that constitute the living body 1.

The light irradiation point 11 has a light emitting element comprising a power supply source and a light source, which is able to be arranged in the interior of the living body 1, for example, by using a capsule or the like.

In addition, it is preferable that the light emitting element be provided with a medium that serves to expand the angle of radiation of light to be emitted.

As such a medium, there can be used a light scattering medium or a movable type mirror.

Moreover, as the light scattering medium, there can be used, for example, spherical frosted or ground glass or the like.

### THIRD EMBODIMENT

Next, reference will be made to a biological information imaging apparatus according to a third embodiment of the present invention in which a contrast medium is introduced into the interior of a living body so as to obtain biological information thereof.

The biological information imaging apparatus of this third embodiment serves to make it possible to image the place of collection, a concentration distribution and the like of the contrast medium introduced into the living body for the diagnosis of various diseases such as tumors, Alzheimer's disease, carotid artery plaques, etc., by the use of the contrast medium.

The biological information imaging apparatus of this embodiment uses a biological information imaging apparatus which is basically identical in construction to that of the first embodiment or the second embodiment except for a feature that a contrast medium is introduced into a living body so as to obtain biological information thereof.

A light irradiation point irradiates pulsed light of a specific wavelength to be absorbed by the contrast medium introduced into the living body.

In addition, acoustic wave detectors for detecting the acoustic waves generated by the contrast medium, which has been accumulated or collected in the living body and has absorbed a part of the light energy, and for converting them into corresponding electric signals are provided outside the living body, as described in the first embodiment or the second embodiment.

Although indocyanine green (ICG), etc., is typically used as the contrast medium, any material can be used which is irradiated by pulsed light to generate an acoustic wave.

### FOURTH EMBODIMENT

Reference will be made to a biological information imaging apparatus according to a fourth embodiment of the present invention.

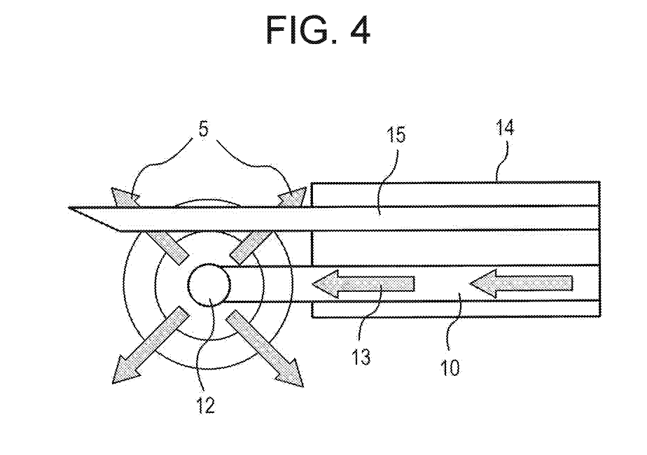

FIG. 4 shows a view for explaining a construction example of the biological information imaging apparatus of this fourth embodiment.

In FIG. 4, reference numeral 14 denotes a catheter, and 15 a puncture needle.

The biological information imaging apparatus of this embodiment is used, when a diseased part such as a tumor or a region with a doubt of a diseased part has been discovered as a result of a diagnosis by imaging, to gather cells for more precise medical examination or to apply the action of injection or the like to the diseased part.

A puncture needle such as an injection needle, a cytologic needle or the like is used for performing such an action. According to the apparatus of this embodiment, it is possible to perform puncturing by using the obtained image while observing the diseased part, so it becomes possible to take an appropriate action.

It is preferable that the point or tip of the puncture needle 15 be fixed in the vicinity of a light irradiation portion. For example, the puncture needle 15 and an optical wave guide such as an optical fiber 10 are preferably received in the catheter 14. In addition, a guide hole for guiding the puncture needle 15 may be provided in the vicinity of the light irradiation portion.

The puncture needle 15 and the light irradiation portion may be completely separated from each other without fixing the tip of the puncture needle 15 in the vicinity of the light irradiation portion, so that they can be inserted into the living body from separate positions.

### FIRST EXAMPLE

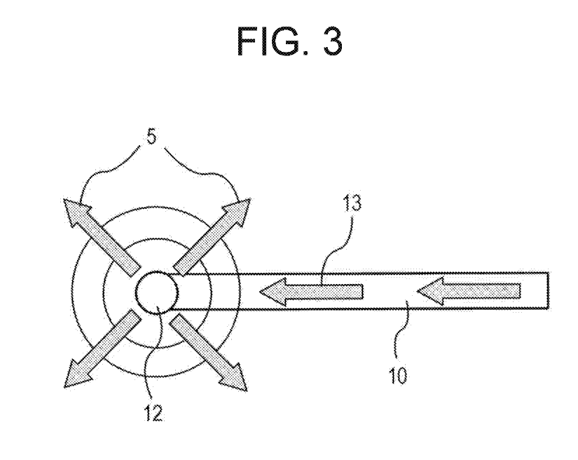

Now, reference will be made, as a first example of the present invention, to an exemplary biological information imaging apparatus for obtaining an absorption coefficient distribution of a tumor in a living body, while using FIG. 1 and FIG. 3.

FIG. 3 is a view explaining an insertion type light source in this example.

In FIG. 3, reference numeral 5 denotes light propagating in a living body, 10 an optical fiber, 12 a light irradiation point in the form of a light scattering medium, and 13 light propagating in the optical fiber 10.

In this example, as a light source, there is used a Q-switched Nd:YAG laser that can oscillate nanosecond pulsed light having a wavelength of 1,064 nm.

The pulsed light generated has a pulse width of about 5 ns and a repeat speed of 10 Hz.

This pulsed light 13 is introduced into the interior of the living body by the use of the optical wave guide in the form of the optical fiber 10, so as to be scattered by the light scattering medium 12, which is formed of spherical frosted or ground glass and is arranged at the tip of the optical fiber 10, whereby it is irradiated to the living body at an angle of radiation equal to or more than 2π steradian.

As an acoustic wave detector 3, there is used a piezo-type transducer having a center frequency of 2.5 MHz, and an acoustic wave issued from the interior of the living body is detected at multiple points by scanning the transducer.

An acoustic wave signal detected by the acoustic wave detector 3 and converted into an electric signal is recorded by an oscilloscope, and is thereafter sent to a computer, and analyzed there.

If measurements are made with a specimen imitating or mimicking a tumor embedded in a soft tissue by using such an apparatus, it is possible to obtain a distribution of optical property values in a deep portion of the living body in a wider range than conventional one.

### SECOND EXAMPLE

Next, reference will be made, as a second example of the present invention, to an exemplary biological information imaging apparatus that is capable of obtaining an absorption coefficient distribution of a tumor in a living body, and performing puncturing by using a distribution image while observing a diseased part, with reference to FIG. 4.

A method of obtaining the absorption coefficient distribution of the tumor in the living body and acquiring the distribution image thereof is similar to that in the above-mentioned first example.

In this second example, an insertion type light source having a puncturing function of FIG. 4 is employed without using the insertion type light source of FIG. 3 in the first example.

FIG. 4 is a view for explaining the insertion type light source having a puncturing function that is used with the biological information imaging apparatus in the second example. An optical fiber 10 having a light scattering member 12 at its tip end and a puncture needle 15 are inserted into a catheter 14, and the optical fiber 10 having the light scattering member 12 and the tip of the puncture needle 15 protrude from one end of the catheter 14.

The catheter 14 is inserted from a skin of the living body, and the optical fiber 10 having the light scattering body 12 at its tip and the puncture needle 15 are inserted into the catheter 14. By doing so, a light irradiation point and the puncture needle 15 are introduced into the interior of the living body.

Then, by irradiating light from the light scattering member 12, the absorption coefficient distribution of the tumor is imaged, and a user can make the needlepoint of the puncture needle 15 reach the tumor while observing it. When the needlepoint reaches the tumor, cells of the tumor can be gathered in an appropriate manner. Also, it becomes possible to appropriately take an action such as injection or the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2007-164654, filed on Jun. 22, 2007, which is hereby incorporated by reference herein in its entirety.

1. A biological information imaging apparatus comprising:
a member that is adapted to be introduced into an interior of a living body and has a light irradiation portion for irradiating light with an angle of irradiation equal to or more than 2π steradian to the interior of said living body; and
a signal detector that is arranged outside said living body and detects a signal output based on light irradiation in the interior of said living body by said member having the light irradiation portion.

2. A biological information imaging apparatus according to claim 1, wherein said signal detector is an acoustic wave detector that detects, as said signal, an elastic wave generated from a light absorber in the interior of said living body which has absorbed a part of the energy of light irradiated from said member having the light irradiation portion.

3. A biological information imaging apparatus according to claim 1, wherein said member having the light irradiation portion is constructed to be connected through an optical wave guide to a light source arranged outside said living body, in such a manner that light from said light source is able to be irradiated from said light irradiation portion by way of said optical wave guide.

4. A biological information imaging apparatus according to claim 1, wherein said member having the light irradiation portion has a puncturing function.

5. A biological information imaging apparatus according to claim 4, wherein said member having the light irradiation portion is provided with a guide hole for guiding a puncture needle.

6. A biological information imaging apparatus according to claim 1, wherein said member having the light irradiation portion is composed of a light emitting element which is adapted to be arranged in said living body and which is provided with a power supply source and a light source for irradiating light to the interior of said living body.

7. A biological information imaging apparatus according to claim 1, further comprising an information processing unit that analyzes said signal detected by said signal detector, and obtains information according to an optical property distribution of said living body.

8. A biological information imaging apparatus according to claim 1, wherein said light absorber in said living body is a tumor or a blood vessel in said living body.

9. A biological information imaging apparatus according to claim 1, wherein said signal detector is constructed in such a manner that it is able to detect said signal in a plurality of locations.

10. A biological information imaging apparatus according to claim 1, wherein said light source is a light source that generates pulsed light.

11. A biological information imaging apparatus according to claim 1, wherein said pulsed light has a wavelength of not less than 400 nm and not more than 1,600 nm.
